(19) United States
(12) Patent Application Publication
(10) Pub. No.: US 2018/0108434 A1
(43) Pub. Date: Apr. 19, 2018

(54) Multi-Application Personal Health Record Microprocessor Card

(71) Applicant: SCS Card Technology Inc., Calgary (CA)

(72) Inventor: Alix Michael Freedman, Calgary (CA)

(73) Assignee: SCS Card Technology Inc., Calgary, AB (CA)

(21) Appl. No.: 15/556,403

(22) PCT Filed: Mar. 10, 2016
(86) PCT No.: PCT/CA2016/000069

§ 371 Date: Sep. 7, 2017

(30) Foreign Application Priority Data
Mar. 10, 2015 (CA) 2,884,544

(51) Int. Cl. G16H 10/65; G06Q 50/22; G06F 21/32; G06K 19/07

(52) U.S. Cl. CPC G16H 10/65; G06K 19/07; G06F 21/32; G06Q 50/22

(57) ABSTRACT

A personal health record card for storing instantly accessible medical information pertaining to the individual associated with the personal health record card is provided. The memory device for storing an individual's personal health information, said memory device comprising: a memory that stores computer readable data, said memory storing: a primary information section comprising information that identifies the individual associated with the memory device and basic information related to the identity of the individual, the primary information accessible by a memory device reader without any authentication; a secondary information section comprising a first level of the individual's sensitive and confidential health information, said secondary information section being accessible only by the memory device reader after authentication; and optionally, a tertiary information section comprising a second level of the individual's sensitive and confidential health information, said tertiary information section being accessible only by the memory device reader after a second level of authentication.

### FIELD OF THE INVENTION

The present invention generally pertains to a portable computer readable memory device such as a personal health record card for storing an individual's medical information.

### BACKGROUND OF THE PRESENT INVENTION

In the world of current-edge technology where most vital information such as banking, and employment are always instantly accessible via smart cards in a wallet, there is a dearth of health information being easily accessible. The move from medical information stored on paper files in doctors' offices to a convenient personal health record card that can be carried everywhere is long overdue.

The potential to be able to carry one's personal medical information at all times has numerous benefits. In case of any emergency or if the individual is unconscious the medical responders will be able to access the vital medical information and basic personal details of the personal health record card user that can immensely help in determining the course of immediate treatment. Such a personal health record card or any portable computer readable memory device can make transferring to a new doctor or physician extremely easy as there is minimal chance of mistakes in reading all updated information on the card. With all of the updated medical information on the personal health record card, the user can have the peace of mind that all of his/her prescriptions from pharmacies, laboratory results, physician and care provider notes and even medical insurance information is all in one highly secured personal health record card that is accessible to multiple authorised users depending on their security clearance.

One key aspect that is crucial for a successful healthcare related personal health record card or any portable computer readable memory device is that it not only stores medical information on the card but that it immediately updates the data on the clinic's Electronic Medical Record (EMR) and the cloud (secure internet storage), hence making the most recent data available offline or online by its seamless integration of technology.

### SUMMARY OF THE INVENTION

According to a first aspect of the present invention, there is provided a memory device for storing an individual's personal health information, said memory device comprising:
a memory that stores computer readable data, said memory storing:
   a primary information section comprising information that identifies the individual associated with the memory device and basic information related to the identity of the individual, the primary information accessible by a memory device reader without any authentication;
   a secondary information section comprising a first level of the individual's sensitive and confidential health information, said secondary information section being accessible only by the memory device reader after authentication; and
   a tertiary information section comprising a second level of the individual's sensitive and confidential health information, said tertiary information section being accessible only by the memory device reader after a second level of authentication.

In another aspect, there is provided a method of using a memory device to provide variable access to information related to an individual associated with the memory device, the method comprising:
providing the individual's personal health record on the memory device, said memory device being operative to store data in a computer readable form, the memory storing:
   a primary information section comprising information that identifies the individual associated with the memory device and non-sensitive medical information related to said individual;
   whereby access to the primary information section requires the use of a card reader; and
   a secondary information section comprising the individual's sensitive and confidential medical information, said secondary information section being accessible by using a card reader and after having entered an authentication code.

In another aspect, there is provided a method of using a memory device associated with an individual to obtain prescription medication, the method comprising:
providing the memory device having a memory operative to store data in a computer readable form, said memory storing:
   a primary information section comprising information that identifies the individual associated with the memory device and his/her non-sensitive basic information, the primary information accessible to a card reader without any authentication; and
   a secondary information section comprising the individual's sensitive and confidential health information, said secondary information section being accessible only after authentication;
taking the memory device to a healthcare professional who first accesses the primary information section by using a memory device reader/writer;
having a healthcare professional input authentication information to access the secondary information section of the memory device;
having a healthcare professional input prescription information in the secondary information section;
providing the memory device to a pharmacist;
having the pharmacist first access the primary information section of the memory device;
having the pharmacist input authentication information to access the secondary information section of the memory device;
having the pharmacist dispense the medication to the individual according to the prescription information input by the healthcare professional in the secondary information section of the memory device.

In another aspect, there is provided a method of using a memory device associated with an individual to obtain laboratory tests information, the method comprising:
providing the memory device having a memory operative to store data in a computer readable form, said memory storing:
   a primary information section comprising information that identifies the individual associated with the memory device and comprises non-sensitive basic information related to the identity of the individual, the primary information being accessible to a memory device reader without any authentication; and
   a secondary information section comprising the individual's sensitive and confidential health information, said secondary information section being accessible by a memory device reader only after authentication;
taking the memory device to a healthcare professional who first accesses the primary information section by using a memory device reader/writer;
having a healthcare professional input authentication information to access the secondary information section of the memory device;
having a healthcare professional input laboratory test requisition information in the secondary information section;
having the individual provide the memory device to an individual representing a laboratory;
having the individual representing a laboratory first access the primary information section of the memory device;
having the individual representing a laboratory input authentication information to access the secondary information section of the memory device;
having the laboratory oversee the testing and result input onto the secondary information section of the memory device by using a card reader/writer;
taking the memory device to a healthcare professional who first accesses the primary information and then after authentication enters the secondary information section where the laboratory test results are reviewed and the individual is informed of the test results.

In another aspect, there is provided a method for using a personal health record card associated with an individual for the latter to obtain a prescription is provided. This method includes:
a healthcare professional accessing the primary information without any authentication, then accessing the secondary information section of the personal health record card after authentication and then entering the prescription information via electronic prescribing (digital Rx) of the medicines ensuring accurate and error free prescriptions. Then the card holder takes the personal health record card to a pharmacy where the pharmacist first access the primary information without any authentication, and then after authentication accesses the prescription information stored in the memory of the personal health record card. The pharmacist then issues a prescription in accordance with the prescription information. The prescription information can also be verified online as there will be a highly secure link available in the section for this purpose.

In another aspect, there is provided a method of using a memory device associated with an individual to assess the individual's compliance with the treatment program, the method comprising:
providing the memory device having a memory operative to store data in a computer readable form, said memory storing:
   a primary information section comprising information that identifies the individual associated with the memory device and comprises non-sensitive basic information related to the identity of the individual, the primary information section being accessible by a memory device reader without any authentication; and
   a secondary information section comprising the individual's sensitive and confidential health information, said secondary information section being accessible by a memory device reader only after authentication;
taking the memory device to a healthcare professional who first accesses the primary information section by using a memory device reader/writer;
having a healthcare professional input authentication information to access the secondary information section of the memory device;
having the healthcare professional review the secondary information section to assess the treatment required by the person;
having the individual undergo treatment;
having the healthcare professional input information related to the treatment session on the secondary information section of the memory device by using a memory device reader/writer;
having the secondary information section of the memory device reviewed by an entity responsible for overseeing the individual to assess the person's compliance with the treatment program.

In another aspect, there is provided a method for using the personal health record card to automatically update all the patient's health records (electronic medical records EMR and electronic health records EHR) when the information is uploaded on the card. This method includes:
a healthcare professional accessing the primary information without any authentication, then accessing the secondary information section of the personal health record card after authentication and then updating the patient's data such as test results, prescriptions, current treatment method, or insurance details. While accessing the information on the personal health record card, the card reader is connected to an online server. This immediately results in the automatic updating of all the information stored in the patient's health records (EMR and EHR) in the doctor's office enabling up to date information on all patient's records.

In another aspect, there is provided a method for using the memory device to determine insurance and payment information details of the individual associated with the memory device in a qualitative and quantifiable manner prior to a medical action, the method comprising:
providing the memory device having a memory operative to store data in a computer readable form, said memory storing:
   a primary information section comprising:
      information that identifies the individual associated with the memory device;
      non-sensitive basic information related to the identity of the individual; and
      insurance information; the primary information section being accessible by a memory device reader without any authentication; and
   a secondary information section comprising the individual's sensitive and confidential health information, said secondary information section being accessible by a memory device reader only after authentication;
taking the memory device to a healthcare provider who can view the insurance information comprising: coverage details of the individual associated with the personal health record card; co-pay information; balance of the benefits; and availability of funds for services and medications prescribed.

In another aspect, there is provided a method for using a memory device to reduce insurance claims by an individual associated with the memory device, the method comprising:
providing the memory device having a memory operative to store data in a computer readable form, said memory storing:
   a primary information section comprising:
      information that identifies the individual associated with the memory device;
      non-sensitive basic information related to the identity of the individual; and
      insurance information; the primary information section being accessible by a memory device reader without any authentication; and
   a secondary information section comprising the individual's sensitive and confidential health information, said secondary information section being accessible by a memory device reader only after authentication;
taking the memory device to a healthcare provider who views the insurance information comprising: coverage details of the individual associated with the personal health record card, by inserting the memory device in a memory device reader, prior to performing a medical action;
having the healthcare provider review the individual's medical history and contact the insurance company to inform that a similar medical action
   was performed recently; and
   having the healthcare provider obtain consent from the insurance company prior to performing the medical action.

### BRIEF DESCRIPTION OF THE DRAWINGS

The invention may be more completely understood in consideration of the following description of various embodiments of the invention in connection with the accompanying drawings, in which:

FIG. 1 is a schematic illustration of a personal health record card according to an embodiment of the present invention;

FIG. 2 is a schematic illustration of a data structure;

FIG. 3 is a systems diagram;

FIG. 4 is a flowchart of a method for having a card issued to a cardholder;

FIG. 5 is a flowchart of a method for using a card to obtain a prescription and laboratory tests requisition;

FIG. 6 is a flowchart of a method for using the card to obtain a prescription from an authorized health care provider;

FIG. 7 is representative screenshot of personal information obtained from the card;

FIG. 8 is a flowchart of a method for using the card at a laboratory to obtain laboratory results;

FIGS. 9A and 9B are flowcharts of a method for using the card to purchase prescribed medication;

FIG. 10 is a systems diagram of a system for use with a smart card to determine if the cardholder is undergoing their court mandated treatment;

FIG. 11 is a flowchart of a method for using a card to determine whether a cardholder is following his/her court mandated treatment.

### DESCRIPTION OF THE PREFERRED EMBODIMENTS

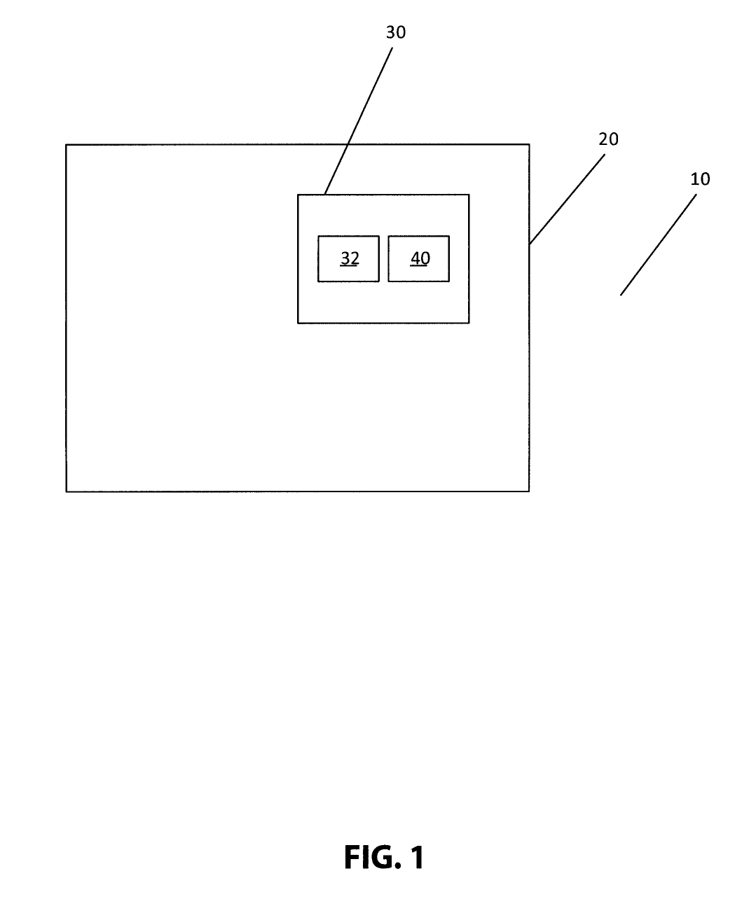

FIG. 1 illustrates a schematic illustration of a personal health record card 10 in accordance with an aspect of the present invention. The card 10 can include a card portion 20 and an integrated circuit chip portion 30 that includes a processing unit 32 and a memory 40. The processing unit can be a microprocessor and the memory 40 can be a computer accessible memory that can be operatively coupled to the processing unit 32.

The card portion 20 can be made of a material such as polyvinyl chloride (PVC) plastic and can be the size of a typical credit card that can fit into a wallet. There can also be a magnetic strip (not shown in FIG. 1) on one side of the card portion 20 for access control; however it is not a necessary part of the card and need not be included in the personal health record card.

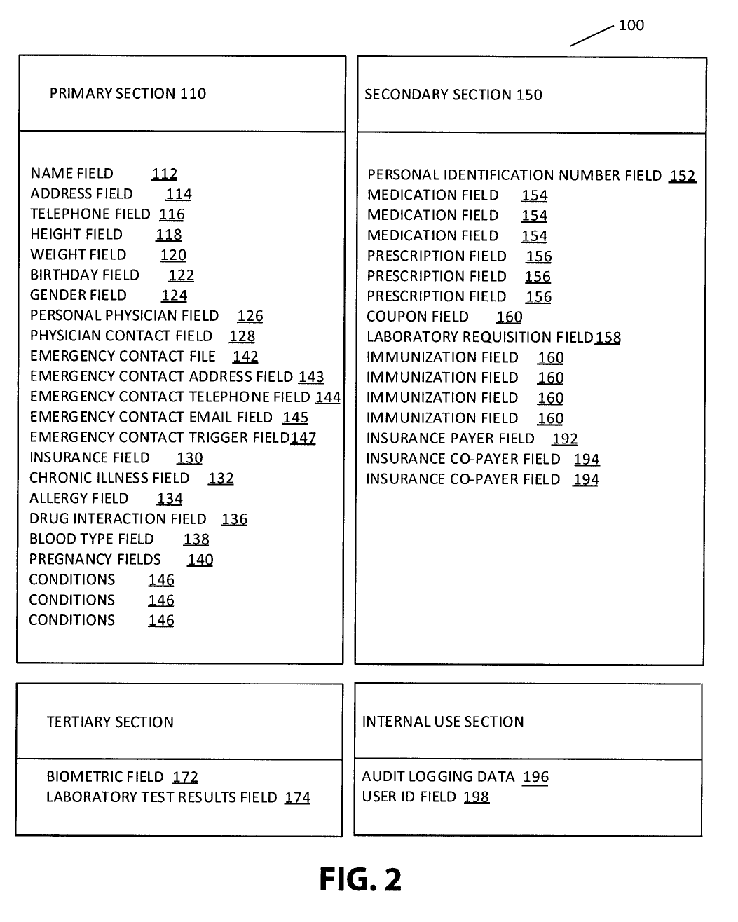

FIG. 2 shows a schematic illustration of a data structure 100 that can be stored in the computer readable memory 40 of the card 10 and used to achieve the methods described here.

The data structure can contain: a primary information section 110; a secondary information section 150; a tertiary information section 170; and an internal use information section 190.

The primary information section 110 can be used to store information that identifies the individual associated with the personal health record card 10 and his/her non-sensitive medical information; and this section is accessible to a card reader without any authentication required. Hence, anyone with access to a card reader can view the primary information section 110 on the personal health record card 10. This aspect is crucial in case of an emergency as the emergency first responders including firefighters, EMTs, police officers, hospital staff, etc. can instantly access the primary information section 110 of the card 10 which can potentially save the life of the individual associated with the personal health record card 10.

A new patient file can easily be opened based on the information in the primary information section 110 of the card as the staff at medical offices (such as dental, optometry, podiatry, laboratory, etc) can access that information when the card 10 is presented to them. This ensures minimal errors in data entry as all the information in the card 10 is entered by a healthcare professional and not by the individual associated with the card his/herself.

In one aspect, the primary information section 110 can consist of a number of different fields for storing information about the individual associated with the card 10. The full name of the individual associated with the card 10 can be stored in the name field 112, and the contact number and home address in the telephone field 116 and address field 114 respectively. Further details describing the card holder's physical appearance such as height and weight can be stored in the height field 118 and weight field 120 respectively. Details such as birth date and gender can be stored in the birthday field 122 and gender field 124 as well respectively. This section 110 can also incorporate information regarding the emergency contact of the individual associated with the card 10 in the emergency contact file field 142, emergency contact address field 143, emergency contact telephone field 144, emergency contact email 145 and emergency contact trigger field 147. Details of the cardholder's personal physician can also be added in the primary information section 110 such as the physician's name in the personal physician field 126 and his/her contact information in the physician information field 128.

Furthermore, the primary information section 110 can be used to store non-sensitive medical information of the card holder-this can include a plurality of chronic illness filed 132 for storing information regarding any major diseases or illnesses the cardholder may suffer from. A plurality of allergy fields 134 can be provided for storing information indicating any allergies the cardholder may suffer from including any allergies to medication. A drug interaction field 136 can also be included in this section 110 for storing information regarding any drug interaction warnings that are applicable to the cardholder. This section 110 can also include a blood type field 138 for storing information about the blood type of the cardholder. A plurality of pregnancy fields 140 can also be included in this section 110 to store information details about the user's pregnancy such as due date and any particular complications, etc. A plurality of condition fields 146 can also be included to store information about any particular health related information that needs to be mentioned here for a correct assessment of the cardholder's health.

The secondary information section 150 can be used to store sensitive medical information that can be accessed only via means of certain authentication. In one aspect, the secondary information section 150 authentication process requires the individual who is accessing the information to provide a personal identification number (PIN). This section 150 can include a personal identification number field 152 which can store the corresponding PIN (a minimum of four alphanumeric characters to a maximum of sixteen alphanumerical characters) that was used to access the secondary information section 150.

The secondary information section 150 can include a plurality of medication fields 154 that the cardholder has been prescribed and this field can also be added in the primary information section if it is non-sensitive and crucial in case of an emergency. The medication fields 154 could be further divided into permanent medications, recent medication and over the counter medications that the cardholder has been prescribed.

The secondary information section 150 of the memory 40 can also be used to store prescriptions written by the physician in digital Rx format (electronic prescriptions) in the plurality of the prescription fields 156. This will include details of the medicine, the amount and dosage required and an identification of the prescribing healthcare professional in these fields 156. In one aspect, the prescription fields 156 can also include an online verification service so the pharmacist who is accessing this information can verify the prescription if needed. A coupon field 160 can be provided to store any coupon information related to the prescriptions that are present in the prescription fields 156. A plurality of immunization fields 160 can also be included in this section 150 indicating the types of immunization the cardholder has had and the date administered.

In one aspect, the secondary information section 150 can include a plurality of laboratory requisition fields 158 that can be used to store information related to the laboratory tests that have been requisitioned by an authorized healthcare professional for the cardholder. The laboratory requisition fields can also include an online secure verification link that can confirm the laboratory tests that have been requisitioned. Additionally, the secondary information section 150 could contain other types of information such as a list of medicines that are kept behind a pharmacy dispensary counter that do not require a prescription, but do require confirmation of some personal information such as a name, address, telephone, age, etc. It could also contain information regarding any prosthetic or orthotic devices prescribed by a healthcare provider to the cardholder.

The secondary information section 150 can also include information regarding the insurance payers in the insurance payer fields 192. These fields 192 can include detailed information identifying the cardholder's medical insurance prescription payer and can include the payment amount expressed as a monetary value and a percentage of the total price. In one aspect, the insurance payer fields can also include further details of the medical insurance of the individual associated with the card 10. This can include but is not limited to, a list of the benefits provided to the cardholder according to the type of insurance plan. In one aspect, the cardholder may have more than one insurance payer and the additional co-payer(s) can be added in the insurance co-payer fields 194. These fields can be used to store information detailing the amount each co-payer has to pay expressed in a monetary value and the percentage of the total price. Preferably, there can also be a link available to an online verification service to confirm the payment details. If the cardholder has to pay a certain amount for the prescription, those details will also be mentioned in this field.

The tertiary information section 170 can be used to store even more sensitive information that requires a higher level of security clearance. In one aspect, the tertiary information section 170 can require biometric data of the individual associated with the card 10. The biometric data can be a constellation of pixels representing a fingerprint of the cardholder. The tertiary information section 170 can include a biometric field section 172 that can provide an authentication of the cardholder's biometric identification. In one aspect, this can be a constellation of pixels representing his/her fingerprint. The cardholder may have to provide his/her fingerprint for the card reader to correctly identify according to the biometric field 172 that is representing the fingerprint of the cardholder, before the card reader can obtain access to the information in tertiary information section 170. The information stored in this section 170 can include privacy protected condition of the cardholder, mental health conditions, special advisories, laboratory results, etc. A plurality of laboratory test result fields 174 can be provided in the tertiary information section 170 where the results can be stored.

An internal use information section can also be provided that can include audit logging data 196 to be used by the provider of the card 10 for troubleshooting, maintenance, etc and a user ID field 198 for use by the card 10 providers to access the internal use information section 190.

In one aspect, when the first responder such as the emergency crew uses a card reader to view the primary information section 110 on the card 10, the information is immediately sent to the home base hospital's PCs where the relevant doctors, nurses and staff can access this information and do the necessary set up for the cardholder to effectively treat him/her. When the information 110 is delivered from the SCS app to the hospital SCS server PCs, a message is shown to the first responders card reader to confirm this.

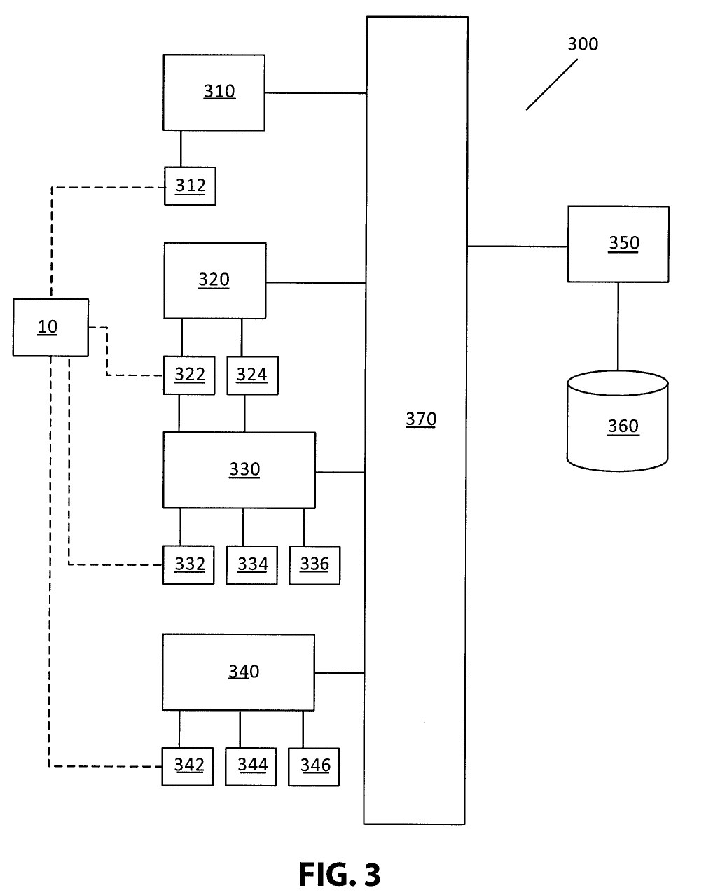

FIG. 3 shows a schematic illustration of a system 300 that can be used with the card 10. The system can include: a cardholder device 310; a medical professional device 320; a laboratory device 330; a pharmacist device 340 and a remote device 350. The aforementioned devices can be local computer systems and can be in communication with the remote device 350 through a network 370.

The cardholder device 310 can be a data processing system such as a personal computer, mobile device, etc. that allows the cardholder to access the remote device 350. The cardholder device 310 can have a program operative for accessing and viewing the information on the card 10. In one aspect, the cardholder device 310 can include a card reader/writer 312 that is operative to read the information from the card 10 and allow the cardholder to view the information stored on the card 10 on the cardholder device 310.

The medical professional device 320 can also be a data processing system, such as a computer, mobile device, etc. that allows a individual using it to access the remote device 350 using the medical professional device 320. Usually, the medical professional device 320 is located at a medical services office. The medical professional device 320 can have a program operative for accessing the information on the card 10 and updating information on the card 10. The medical professional device 320 can have a card reader/writer 322 to allow the information on the card to be accessed and obtained by the medical professional device 320 as well as write new information to the memory 40 of the card 10. Additionally, the medical professional device 320 can have a PIN pad 324 allowing an individual to enter a personal identification number (PIN) into the PIN pad 324 and thereby the medical professional device. Alternatively, the personal identification number (PIN) could be entered using a standard keyboard attached to the medical professional device 320.

The laboratory device 330 can be located at a laboratory site where laboratory tests are performed on the cardholder and it can be a data processing system such as a computer, etc. The laboratory device 330 can have a program operative for accessing the information on the card 10 and updating information on the card 10. The laboratory device 330 can include a card reader/writer 332 to read and write information from and to the card 10. In one aspect a PIN pad 334 and a fingerprint reader 336 can be provided to allow the cardholder to be authenticated.

The pharmacist device 340 can be located at a pharmacy where a prescription can be issued and can be a data processing system such as a computer, etc. The pharmacist device 340 can have a program operative for accessing the information on the card 10 and updating information on the card 10. The pharmacist device 340 can include a card reader/writer 342 to read and write information to and from the card 10 and in one aspect can include a PIN pad 344 and a fingerprint reader 346 to allow the cardholder, and the card 10 to be authenticated if required.

The remote device 350 can be in operating communication with all of the other devices 310, 320, 330 and 340 over the network 370. The remote device 350 can also be operably connected to a database 360. The remote device 350 can be accessible by the patient's physician, health provider, hospital physicians, hospital nurses, etc. It can securely store patient information and laboratory results related to the patient in the database 360.

The network 370 can be a local internet or even a public network such as the Internet over which all this information is shared securely.

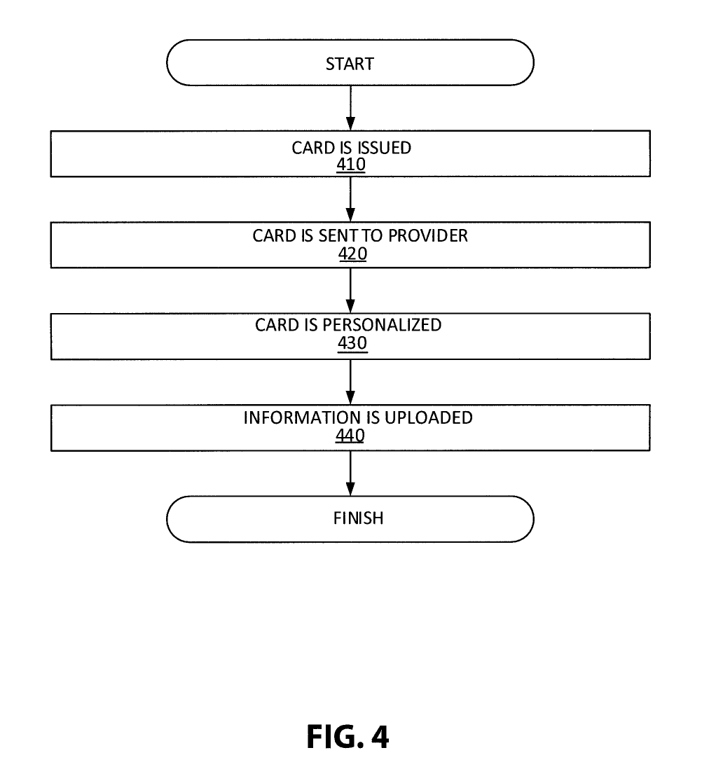

FIG. 4 illustrates a flowchart of an overview of a method to obtain a card 10. At step 410 the card can be issued to the specified person. The card can be created by the provider and a record stored in the database 360 corresponding to this new card. At step 420 the new card 10 can be sent to the provider who will be providing it to the cardholder. At step 430 the card 10 can be personalized. The provider can obtain personal information from the cardholder, such as his or her name, address etc. and this information can be taken and written into the proper fields in the primary information section 110. At this time, the cardholder can also give to the provider the name of his or her personal physician or clinic to be added to the personal physician field 126 and the physician contact field 128. In this manner, personal information can be stored to the card 10. The personal information can be verified by checking the potential cardholder's ID cards and current medical records obtained from his/her physician.

Alternatively, the provider can download all of this information from an existing database such as an Electronic Health Record or Electronic Medical Record enterprise-sized software system in advance of providing the card 10 to the cardholder.

At this point the cardholder can be prompted to insert a PIN number which can then be stored in the personal identification number field 152 and a fingerprint or other biometric indicator can be taken of the cardholder and stored in the biometric field 172. This information can then be later used to authenticate the cardholder when the card 10 is in use.

Once the personal information has been stored on the card 10, this information stored on the card at step 430 can be uploaded to the remote device 350 and the database 360 at step 440 and the method can end.

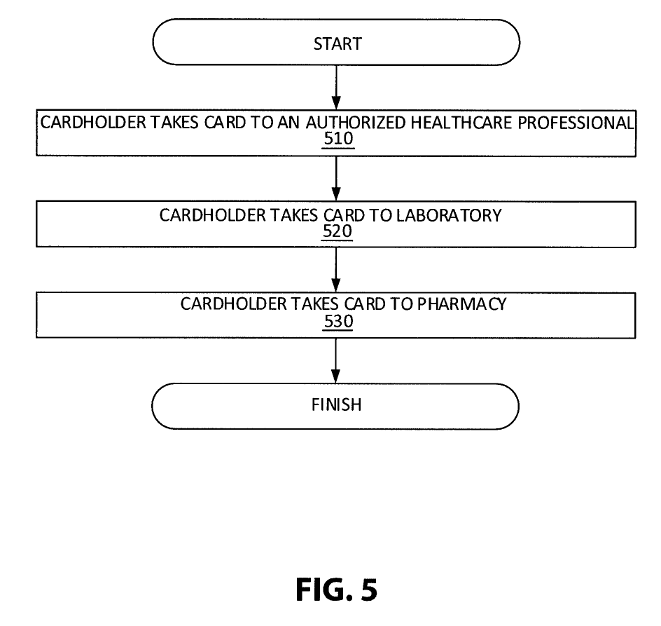

FIG. 5 illustrates a flowchart of a method for using the card 10 to obtain a prescription and/or requisition laboratory tests. In the method, the card 10 is used to obtain a prescription and/or requisition laboratory tests from a medical professional such as a doctor. Then the individual associated with the card 10 takes the card 10 to the laboratory test location where the laboratory tests are performed on the cardholder. Subsequently, the patient takes the test results to the doctor who reads and interprets the results before issuing a prescription. The doctor than inputs a prescription onto the card 10 and the individual associated with the card 10 takes the card 10 to the pharmacist who fills the prescription and pays a portion of the prescription, if any, according to the cardholder's insurance.

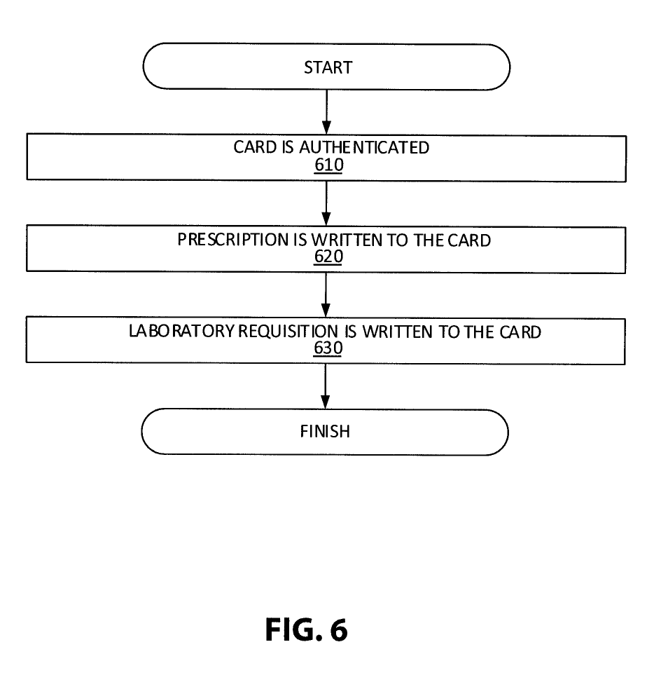

FIG. 6 is a flowchart of a method for what occurs with the card 10 during step 510 of the method shown in FIG. 5. The method begins with the cardholder providing his/her card 10 to the authorized health care provider and the authorized health care provider authenticating the card 10 at step 610. To authenticate the card 10, the health care provider can insert the card 10 in the card reader/writer 332 connected to the medical professional device 320. Using the card reader/writer 332 and the medical professional device 320, the health care provider can obtain access to the information stored in the primary information section 110 of the memory 40 of the card 10 by simply using the card reader/writer 332 to view the contents of the card 10. The information in the primary information section 110 can then be displayed on a screen of the medical professional device 320.

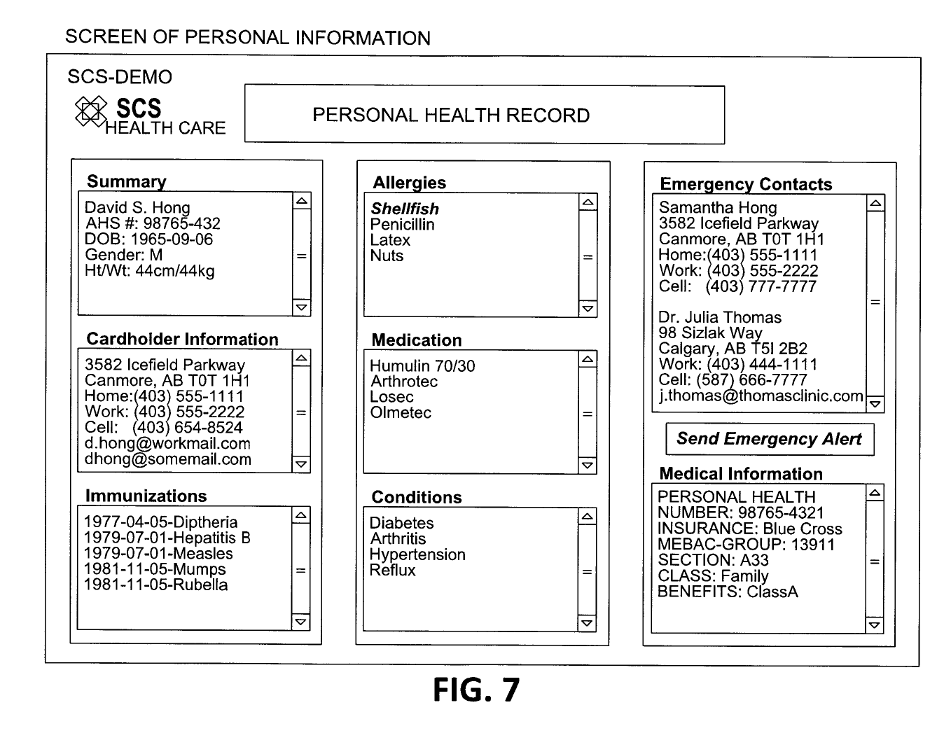

FIG. 7 illustrates a representative screenshot of how the information in the primary information section 110 of the memory 40 of the card 10 can be displayed.

In addition to the information in the primary information section 110 of the memory 40 of the card 10, the authorized healthcare provider will also need to have access to the secondary information section 150 of the memory 40 of the card 10. In order to authenticate the cardholder and obtain access to the secondary information section 150, the cardholder will have to provide access to this section. In one aspect, this can be providing the PIN that matches the PIN indicated in the personal identification number field 152 of this secondary information section 150 of the memory 40. In one aspect, the PIN can be entered into a PIN pad 324 connected to the medical professional device 320.

The card 10 will then be authenticated and the authorized healthcare provider will have access to the information in the primary information section 110 and the secondary information section 150 of the card 10.

Once the card 10 has been authenticated at step 610, a prescription can be written to the card at step 620. The card reader/writer 322 and the medical professional device 320 can be used to access the secondary information section 150 of the memory 40 of the card 10 and access one of the prescription fields 156 in this section. The card reader/writer 332 can then be used to write information identifying the prescription being issued by the authorized healthcare provider to the prescription field 156. In one aspect, this information can include medication prescribed, the amount prescribed and an identification of the prescribing medical professional.

At step 630 a laboratory requisition is written to the card. The authorized healthcare provider will use the medical professional device 320 and the card reader/writer 322 to access the secondary information section 150 of the memory 40 of the card 10 and write a laboratory requisition to a laboratory requisition field 158. This laboratory requisition can identify the types of laboratory tests desired by the medical professional issuing the prescription.

Although step 620 and 630 are shown sequentially in FIG. 6, they could be performed in different visits to an authorized healthcare professional.

In one aspect, the requisition laboratory tests could be diagnostic tests using a biomarker.

After the lab requisition is written to the card at step 630, the method can end and referring to FIG. 5, the method can move on to step 520 with the cardholder taking the card 10 to a laboratory to have the laboratory tests performed that were written to the card 10 in step 510.

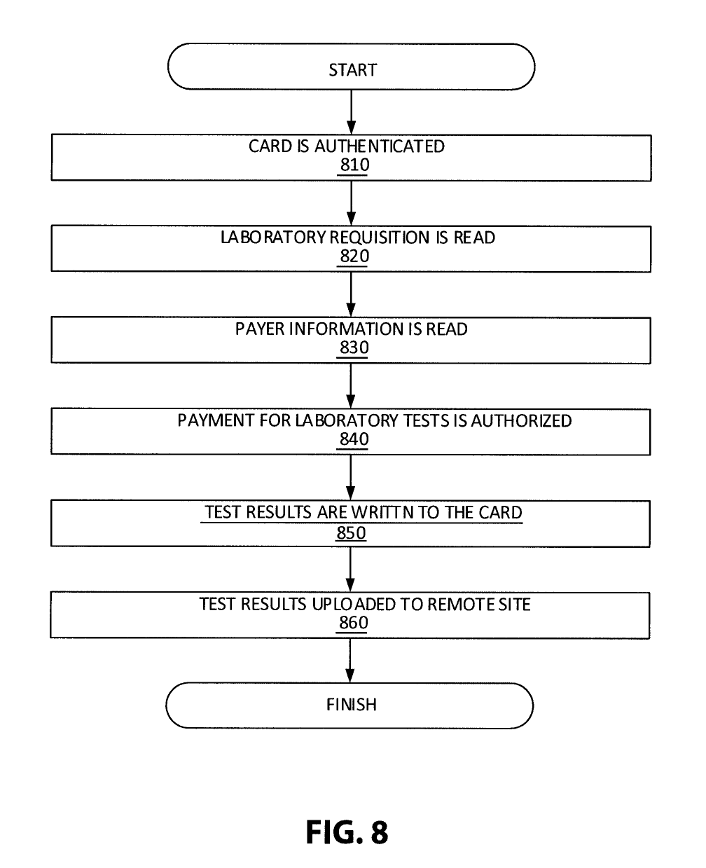

In FIG. 8, a flowchart of a method is shown for using the card 10 to obtain laboratory results requested by an authorized healthcare provider. The method can start at step 810 when the card 10 can be authenticated. Personnel at the laboratory can insert the card 10 into the card reader/writer 330 of the laboratory device 332. Without any authentication, the laboratory device 330 can access the information stored in the primary information section 110 of the memory 40 of the card 10. To access the information in the secondary information section 150 of the memory of the card 10 (where the laboratory requisition is stored), the cardholder will have to provide a PIN or other authenticating information to allow access to the information in the secondary information section 150 of the memory 40 of the card 10. In one aspect, this PIN could be entered by inputting it into a PIN pad 334 connected to the laboratory device 330. As the results of the laboratory results can be saved in the tertiary information section 170 of the memory 40, the authentication of the card holder and the card 10 at step 810 may require additional authentication for access to the tertiary information section 170 to be obtained by the laboratory device 340. In one aspect, this additional authentication could require the cardholder to provide his/her fingerprint on the fingerprint reader/writer 336 connected to the laboratory device 330.

The fingerprint obtained can be matched to the constellation of pixels in the biometric field 172 of the tertiary information section 170 and, if it matches, allow the laboratory device 330 access to the tertiary information section 170 of the memory of the 40 of the card 10. Once the card 10 and cardholder are authenticated at step 810, the laboratory requisition stored in the laboratory requisition fields 158 in the secondary information section 150 of the memory 40 of the card 10 can be accessed by personnel at the laboratory using the laboratory device 330 and the types of laboratory tests requisitioned by the authorized healthcare provider can be determined at step 820.

At step 830 the payer information for the tests can be determined from the card 10. The information in the insurer payer field 192 can be accessed and this information used to determine which parties are paying for the laboratory tests and the amount that is being covered. If there are one or more co-payers, this information can also be obtained from the insurance co-payer fields 194.

At step 840 payment for the laboratory tests can be authorized using the payer and co-payer information obtained from the card 10 at step 830.

Once the payment is authorized, the laboratory can proceed to perform the tests outlined in the laboratory requisition read off the card 10 at step 820 and the necessary or requested laboratory tests performed on the cardholder by the laboratory. In one aspect, these tests can be diagnostic tests to determine the presence or levels of a biomarker in the body of the cardholder.

Once the laboratory tests have been performed, if the results are obtained relatively quickly, at step 850 the test results can be written to the test result fields 174 in the tertiary information section 170 of the memory 40 of the card 10 if the cardholder and the card 10 are still present at the laboratory.

At step 860 the test results can be uploaded to the remote device 350. The test results can be transmitted from the laboratory device 330 over the network 370 to the remote device 350. At the remote device 350 these laboratory results can be saved to the database 360. These files can be encrypted when they are sent for security reasons. In one aspect, the laboratory test results can be transmitted to the medical professional device 320 for viewing by the authorized medical professional. Once the lab results are uploaded to the remote site 350, the method can terminate.

Referring again to FIG. 5, once the cardholder has used his/her card 10 at the laboratory at step 520 to obtain the requisitioned laboratory tests, the cardholder can take the card 10 to a pharmacy and use it to obtain and pay for the prescribed medication at step 530.

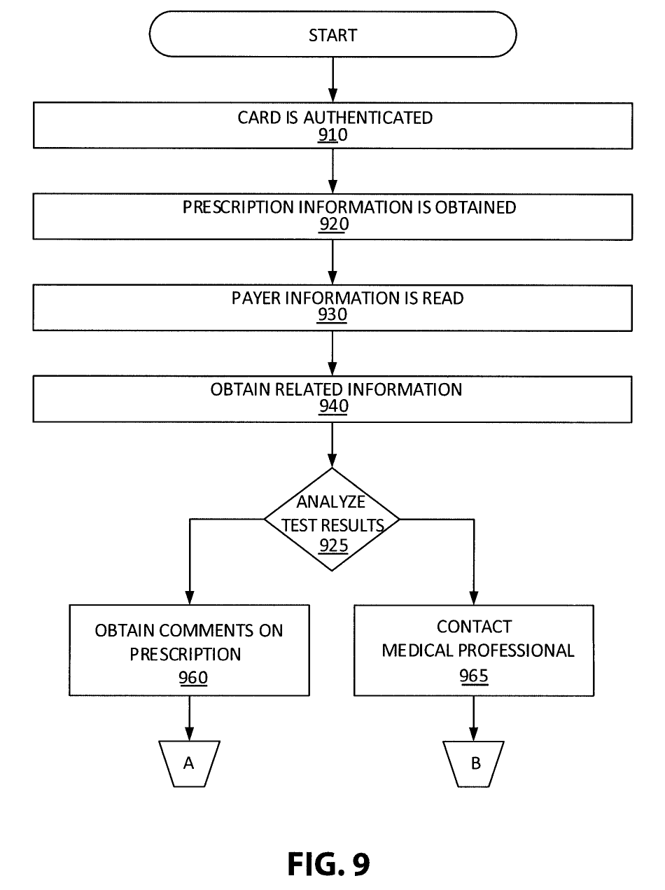

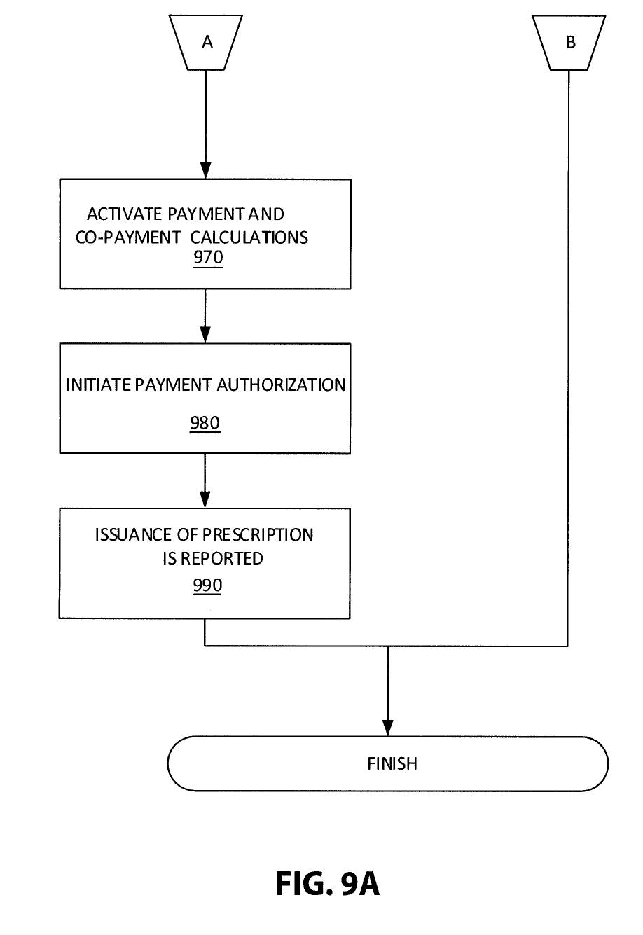

FIG. 9 illustrates a flowchart of a method of the card 10 being used at a pharmacy to obtain a prescribed medication. Method starts at step 910 the card 10 of the cardholder can be authenticated. The pharmacist can insert the card 10 into the card reader/writer 342 of the pharmacy device 340. Since the information about the prescription is saved in the secondary information section 150 of the memory 40 of the card 10 and the laboratory results may be saved in the tertiary information section 170 of the memory 40 of the card 10, the authentication of the card 10 at step 910 can require the cardholder to provide both his/her PIN and a fingerprint to allow both of these sections of the memory 40 to be accessible to the pharmacy device 340.

With the cardholder and the card 10 authenticated at step 910, at step 920 detailed prescription information (such as amount of medication to provide, the dosage dose and the name of the healthcare provider who prescribed the prescription) can be obtained from the card 10 by the pharmacist.

Optionally, if the prescription fields 156 include information indicating a link to a website to have the prescription authorized, the pharmacist device 340 can use the link and the information in the prescription fields 156 to authenticate the prescription. The links can be links to the remote device 350 which can be used to authenticate the prescription.

At step 930 the payer information is obtained from the card 10. The pharmacist device 340 can access the secondary information section 150 of the memory 40 of the card 10. The information in the insurance payer field 192 can be accessed and this information can be used to determine which parties are paying for the laboratory tests and how much is being covered. If there are one or more co-payers, this information can also be obtained from the co-payer fields 194 at step 940.

At step 950 the lab results can be obtained to allow the pharmacist to ensure that the medication indicated by the prescription can be issued. The pharmacy device 340 and the card reader/writer 342 can access the tertiary information section 170 of the memory 40 and obtain the lab results stored in the laboratory test results field 174. With these lab results, the pharmacist can determine if there is anything in the lab results that would change his/her decision about issuing the prescription at step 955. If the laboratory tests were for a biomarker, the pharmacist can determine whether or not the test results for the biomarker indicate that the medication should be prescribed.

If at step 955 it is determined that the test results indicate that the medication indicated by the prescription can be issued, then the method can move on to step 960 and obtain any comments on the prescription made by the authorized healthcare provider.

After step 960, the method can move on to step 970 and the payment and co-payment calculations can be initiated. The payer and co-payer information obtained at step 930 can be used and applied to the price of the medication to fill the prescription. For the payer and each co-payer, the amount each payer and co-payer will pay of the total price of the medication being purchased can be calculated using the information in the insurance payer field 192 and the insurance co-payer field 194. At step 980 payment authorization can be obtained for the medication from the insurance payer and co-payer. With the payment of the prescribed medication, the pharmacist can provide the medication to the cardholder and at step 990 the issuance of the prescription can be reported by the pharmacist. To report the issuance of the prescription, the pharmacist device 340 can transmit the issuance of the prescription to the remote device 350 so that the healthcare professional, the cardholder and other interested parties can log on to the remote device 350 and see that the prescription has been issued and completed.

Referring again to step 955, if at this step it is determined that the laboratory test results do not support the prescription issued by the medical health professional, the method can then move on to step 965. At step 965, the pharmacist can contact the authorized healthcare provider with care alternatives and the method can end with the pharmacist not prescribing the cardholder with any medication. In this manner, the method can prevent medication from being issued to the cardholder that may not be ideal or even hazardous based on the test results.

In a further aspect of the present invention, the card 10 can be used in conjunction with a court mandated treatment program. These programs typically require the individual to either abstain from taking an illegal or other substance during a probationary period or the individual could be required to undertake some medication or vaccination program as part of their treatment. For example, there are some vaccinations now available that prevent the effects of some substances, such as heroin, from affecting a individual who has undergone the vaccination program.

The main issue with these programs is the necessary monitoring of the individual to ensure that they are following the treatment program. This usually requires that the individual presents him/herself at a specific location to be tested for the presence or the lack of the substance in question. The card 10 can be used to record and report these tests results.

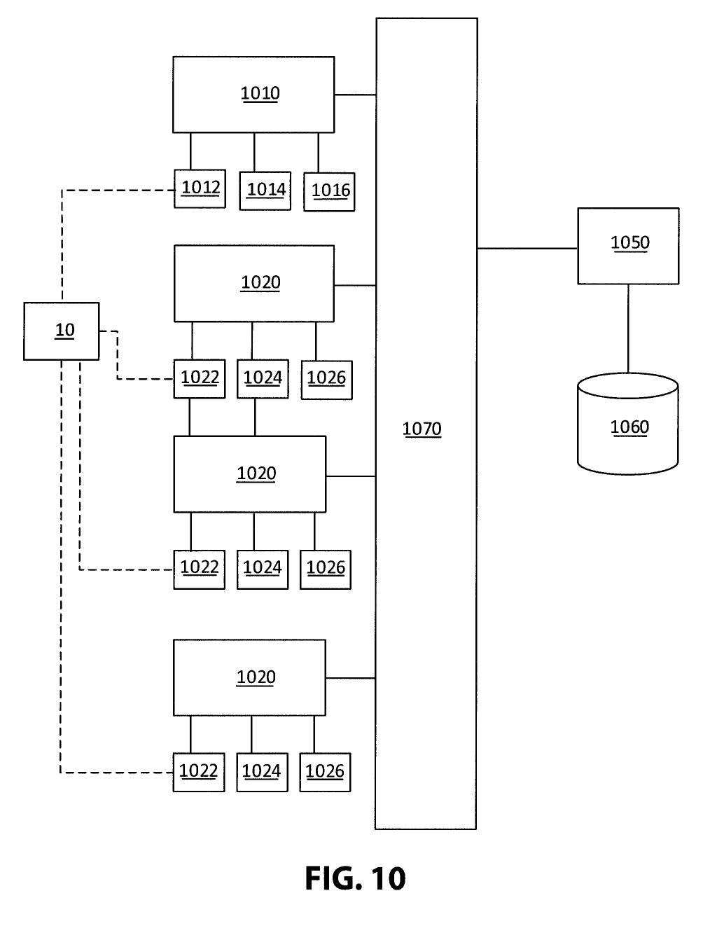

FIG. 10 illustrates a system 1000 for implementing a method of testing and monitoring whether a cardholder is complying with a court mandated treatment program. The system 1000 comprises of a laboratory device 1010, a plurality of kiosk devices 1020 and an authority's device 1050.

The laboratory device 1010 can be located at a laboratory site where laboratory tests can be taken of the cardholder. The laboratory device 1010 can be a data processing system such as a computer, etc. and it can include a card reader/writer 1012 to read and write information from and to the card 10 and in one aspect can include a PIN pad 1014 and a fingerprint reader 1016 to allow the cardholder to be authenticated.

The plurality of kiosks 1020 can be government controlled data processing systems that are provided in government buildings or other public areas that are relatively accessible to the cardholders. Each kiosk can have a card reader/writer 1022 for accepting the card 10 of the cardholder and a PIN pad 1024 to allow the cardholder to be authenticated. The plurality of kiosks 1020 can be in communication with the authority's device 1050 over a network 1070 such as the internet.

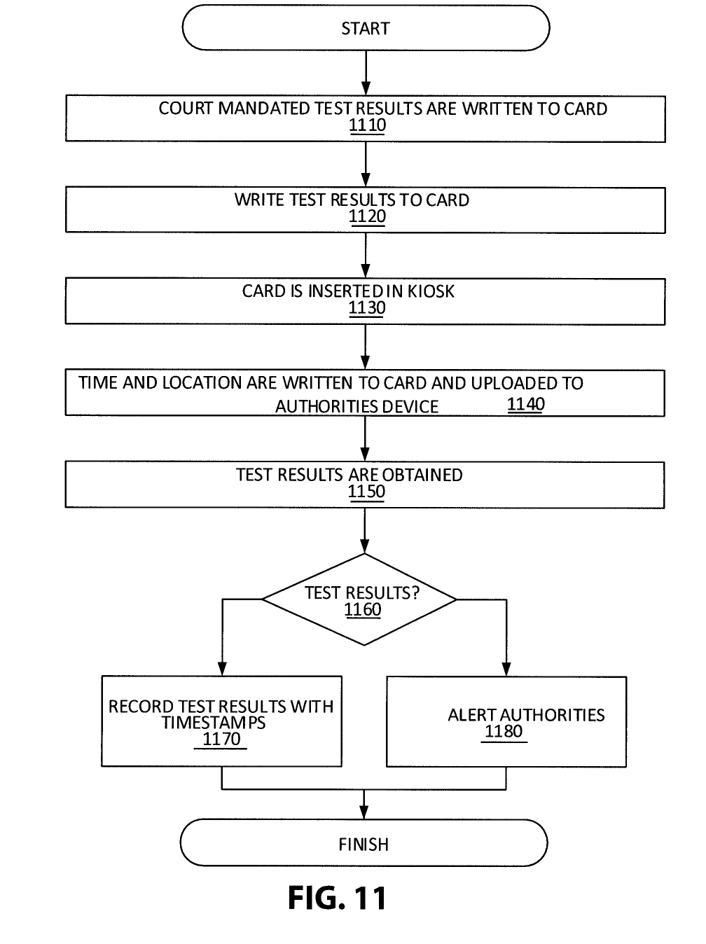

FIG. 11 illustrates a method for determining whether a cardholder is maintaining his court mandated therapy. The method can be initiated with the cardholder taking the card 10 to a laboratory for court-mandated tests at step 1110. These laboratory tests could be urinalysis, blood analysis, etc.

Once the necessary tests are performed, the test results can be written to the memory 40 of the card 10. In one aspect, these test results can be encrypted when they are stored in the memory 40 of the card. After the cardholder has visited the laboratory and the laboratory test results have been stored to the memory 40 of the card 10 at step 1120, the cardholder can visit one of the kiosks 1020 at step 1130. The cardholder can insert his/her card 10 into kiosk 1020 and authenticate themselves.

Once the card 10 has been authenticated the time and location of the access to the kiosk 1150 can be determined at step 1140. The timestamp and location indicator be stored in the memory 40 of the card 10 and uploaded to the authorities' device 1150 to be stored in a database 1060. Hence, the time arid location where the cardholder has accessed a kiosk 1050 can be stored for the proper authorities to view. At step 1150 the laboratory tests stored on the card 10 can be accessed and obtained by the kiosk 1150. At step 1160 the laboratory test results can be analyzed to determine whether the cardholder has passed or failed the tests. If the purpose of the laboratory tests were to determine whether a substance was present in the body of the cardholder that was not supposed to be there (i.e. an illegal substance) and it is found that it is present, then this would be indicated on the card as a failure of the tests. Alternatively, if the tests were to ensure the cardholder was taking a specific medication or vaccination, the lack of this substance showing up in the tests indicates a failure.

If at step 1160 it is determined that the cardholder has failed the tests, then the method can move on to step 1180 and the test results can be written to the memory of the card 10 and uploaded to the authorities device 1050 to be saved in the files related to the cardholder in the database 1060. An alert can also be issued to the cardholder's files and a message will appear on the kiosk computer screen advising the cardholder to contact a specified agency for more information. The alert may be upgraded within an agency's IT system to immediately advise a specific individual or office. Once this alert has been issued at step 1180, the method can end.

However, if at step 1060 it is determined that the cardholder has passed the tests (i.e. no illegal substance are present or the required substance is present) then the method can move on to step 1070 and the approval of the test results and the timestamp can be saved to the memory of the card 10 and the method can end.

The aforementioned detailed description is considered as illustrative only of the principles of the invention. The invention should not be constricted to the exact description and operation shown and described in this patent, as many adaptations and changes can be brought to this memory device to those skilled in the art. Consequently, any such applicable changes or modifications in structure or operation are intended to fall within the scope of the claimed invention of the memory device.

1. A memory device for storing an individual's personal health information, said memory device comprising:
a memory that stores computer readable data, said memory storing:
a primary information section comprising information that identifies the individual associated with the memory device and basic information related to the identity of the individual, the primary information accessible by a memory device reader;
a secondary information section comprising a first level of the individual's sensitive and confidential health information, said secondary information section being accessible only by the memory device reader after authentication; and
optionally, a tertiary information section comprising a second level of the individual's sensitive and confidential health information, said tertiary information section being accessible only by the memory device reader after a second level of authentication.

2. The memory device of claim 1 wherein all the information on the personal health record card is entered by a healthcare professional having an authentication code.

3. The memory device of claim 1 wherein the primary information section comprises the full name, contact number and address of the individual associated with the memory device.

4. The memory of device of claim 1 wherein the primary information section consists of the emergency contact information for the individual.

5. The memory device of claim 1 wherein in the primary information comprises basic information selected from the group consisting of: gender; birthdate; height and weight of the individual.

6. The memory device of claim 1 wherein the primary information comprises critical health related information of the individual selected form the group consisting of:
allergies; and medications being taken by the individual.

7. The memory device of claim 1 wherein the secondary information can only be accessed through authentication method such as a personal identification number (PIN).

8. The memory device of claim 1 wherein the secondary information section comprises prescription information.

9. The memory device of claim 1 wherein the secondary information section comprises laboratory requisition information.

10. The memory device of claim 1 wherein the tertiary information section comprises detailed lab result information.

11. The memory device of claim 10 wherein the additional authentication required to access the tertiary information section is different from the authentication required to access the secondary information section.

12. The memory device of claim 11 wherein the additional authentication required to access the tertiary information section is biometric information of the individual associated with the memory device

13. The memory device of claim 12 wherein the biometric information comprises a fingerprint of the individual associated with the memory device.

14. A method of using a memory device to provide variable access to information related to an individual associated with the memory device, the method comprising:
providing the individual's personal health record on the memory device, said memory device being operative to store data in a computer readable form, the memory storing:
a primary information section comprising information that identifies the individual associated with the memory device and non-sensitive medical information related to said individual;
whereby access to the primary information section requires the use of a card reader; and
a secondary information section comprising the individual's sensitive and confidential medical information, said secondary information section being accessible by using a card reader and after having entered an authentication code.

15. The method of claim 14 wherein the authentication code to access the secondary information section is a personal identification number (PIN).

16. The method of claim 14 wherein the memory of the memory device further comprises a tertiary information section which comprises information related to laboratory results, and which tertiary information section is accessible by using a card reader after having entered a second authentication code.

17. The memory device of claim 16 wherein the second authentication required to access the tertiary information section is different than the authentication code required to access the secondary information.

18. The memory device of claim 17 wherein the additional information authentication is biometric information of the individual associated with the memory device.

19. The memory device of claim 18 wherein the biometric information is a fingerprint of the individual associated with the memory device.

20. A method of using a memory device to obtain prescription medication, the method comprising:
providing the memory device having a memory operative to store data in a computer readable form, said memory storing:
a primary information section comprising information that identifies the individual associated with the memory device and his/her non-sensitive basic information, the primary information accessible to a card reader without any authentication; and
a secondary information section comprising the individual's sensitive and confidential health information, said secondary information section being accessible only after authentication;
taking the memory device to a healthcare professional who first accesses the primary information section by using a memory device reader/writer;
having a healthcare professional input authentication information to access the secondary information section of the memory device;
having a healthcare professional input prescription information in the secondary information section;
providing the memory device to a pharmacist;
having the pharmacist first access the primary information section of the memory device;
having the pharmacist input authentication information to access the secondary information section of the memory device;
having the pharmacist dispense the medication to the individual according to the prescription information input by the healthcare professional in the secondary information section of the memory device.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)
